(19) United States
(12) Patent Application Publication
(10) Pub. No.: US 2024/0108809 A1
(43) Pub. Date: Apr. 4, 2024

(54) Insulin Self-Injection Pen

(71) Applicant: Carol Ann Metz, Colorado Springs, CO (US)

(72) Inventor: Carol Ann Metz, Colorado Springs, CO (US)

(21) Appl. No.: 18/477,365

(22) Filed: Sep. 28, 2023

Related U.S. Application Data
(60) Provisional application No. 63/377,663, filed on Sep. 29, 2022.

(51) Int. Cl. A61M 5/315; A61M 5/32

(52) U.S. Cl. CPC A61M 5/31548; A61M 5/3287; A61M 2202/0486; A61M 2205/587

(57) ABSTRACT

The present invention is a self-injection pen. The self-injection pen has a needle, a medication cartridge, and a pen body. The needle and the medication cartridge are standard commercially available components that are suitable for injecting a liquid medication into the subcutaneous tissue layer of a human or animal body. The medication cartridge has a dose adjustor, and the pen body has an outer casing, a dose adjustment dial and an injector button. The needle is secured to the medication cartridge and allows a liquid medication to flow from the medication cartridge into the body of the user. The dose adjustor controls the amount of liquid medication being administered from the medication cartridge.

### FIELD OF THE INVENTION

The present invention relates generally to a self-injection pen. More specifically, the present invention is a self-injection pen with a dose adjustment dial 32 that is phosphorescent. The phosphor material radiates light, which allows the user to view and set the medication dosage during the day, night, or in low-light conditions.

### BACKGROUND OF THE INVENTION

Today, there are over 37 million people living with diabetes. Diabetes is a chronic health condition that affects how the human body converts food into energy. In particular, the body either does not produce enough insulin (Type 1 diabetes) or the body does not respond to its own insulin (Type 2 diabetes). Insulin is needed to regulate the amount of blood sugar into the body's cells for use as energy. Although there is no cure for diabetes, it can be managed by taking supplemental insulin. Unlike other medications, insulin does not come in pill form because the digestive system would break it down before it has a chance to work. Instead, insulin is commonly administered by injection one to four times per day. For convenience, many people with diabetes prefer self-injection pens. Unlike a typical syringe, a self-injection pen is pre-filled with insulin and has a dose adjustment dial on the end of the handle. This allows the user to select the desired dosage before administering the shot of insulin. More specifically, the user rotates the dose adjustment dial until the desired dosage is visible in the dosage window. Once the dosage is set, the user pushes the needle 1 into the skin and presses the injector button, which then injects the insulin into the body at the proper dosage. However, setting the correct dosage can be difficult at night or in low-light conditions. Most manufacturers of self-injection pens use contrasting colors for displaying dosage (e.g., black background with white numbers). This works well in the daytime, but at night, the numbers can be difficult to read and often requires the user to turn on a light or move into a well-lit area to set the correct dosage. What might seem like a minor inconvenience can become a tiresome task for users who routinely use a self-injection pen at night. Unfortunately, many individuals attempt to provide themselves with injections in the dark and simply hope that they have provided themselves with the correct dose of medication. In many instances, not moving to well-lit areas for a self-injection is a matter of convenience. However, other times it is unavoidable such as during a loss of power during a weather event, a concert, a theater, camping, etc. By providing the user with a device that can be read during low light or no light conditions, the user can ensure that they provide themselves with an injection that always has the proper dose of medicine.

An objective of the present invention is to provide a solution to the aforementioned problem. More specifically, it is an aim of the present invention to provide a self-injection pen with dosage indicator numbers that are always visible, even at night. The present invention is a self-injection pen, wherein the outer surface of the dose adjustment dial contains a phosphor substance. The phosphor substance radiates visible light, giving the dose adjustment dial a “glow in the dark” effect. Also contained on the dose adjustment dial are dosage indicator numbers written in black ink. The color contrast between the phosphor background and the black indicator numbers enables the user to operate the self-injection pen without any external light source. Alternatively, the outer surface of the dose adjustment dial can be painted black, and the dosage numbers are imprinted with a phosphor substance. In either configuration, the glow effect is readily seen through the dosage window. As the user rotates the dose adjustment dial, the dosage numbers cycle through the dosage window, which allows the user to select the desired dosage at any time of the day, with or without light. For night conditions in particular, the user does not need to search for a flashlight or turn on a light to read the dosage number. In a third configuration, a light is integrated into the present invention. Specifically, a small LED light is inserted into one side of the dosage window, which illuminates the dosage indicator numbers when the light is turned on.

### BRIEF DESCRIPTION OF THE DRAWINGS

FIG. 1 is a perspective view of the present invention.

FIG. 2 is a front view of the present invention.

FIG. 3 is a rear view of the present invention.

FIG. 4 is a left-side view of the present invention.

FIG. 5 is a right-side view of the present invention.

FIG. 6 is a top view of the present invention.

FIG. 7 is a bottom view of the present invention.

### DETAIL DESCRIPTIONS OF THE INVENTION

All illustrations of the drawings are for the purpose of describing selected versions of the present invention and are not intended to limit the scope of the present invention.

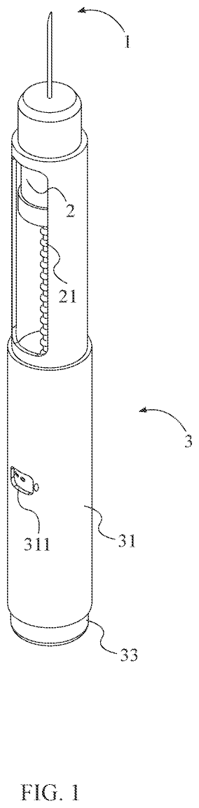

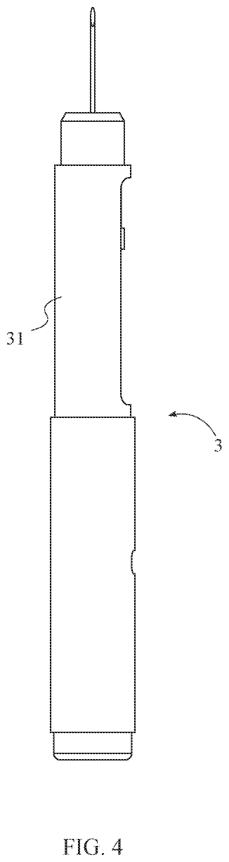

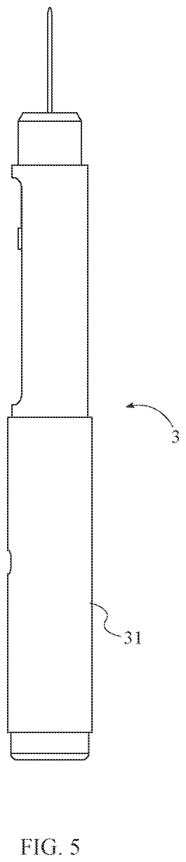

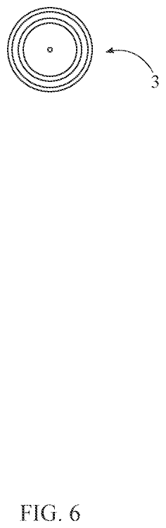

The present invention is a self-injection pen as seen in FIG. 1-7. The present invention comprises a needle 1, a medication cartridge 2, and a pen body 3. The needle 1 and the medication cartridge 2 can be of any type that is commercially available and suitable for injecting a liquid medication into the subcutaneous tissue layer of a human or animal body. The medication cartridge 2 comprises a dose adjustor 21. The pen body 3 comprises an outer casing 31, a dose adjustment dial 32 and an injector button 33. The needle 1 secures to the distal end of the medication cartridge 2. The medication cartridge 2 is positioned within the pen body 3. The pen body 3 encompasses the medication cartridge 2 and the needle 1. The needle 1 extends outwards from the pen body 3. The needle 1 is a long slender cylindrical shape. The needle 1 transfers a liquid medication into the body of a user.

As can be seen in FIG. 1, the proximal end of the medication cartridge 2 is attached to the pen body 3. As a result, the medication cartridge 2 is secured to the pen body 3. The medication cartridge 2 is interchangeable within the pen body 3. Consequently, the medication cartridge 2 can hold various types of liquid medication that can be switched for one another based on the needs of the user. The medication cartridge 2 is secured within the pen body 3. The dose adjustor 21 mechanically adjusts the quantity of medication injected into the body of the user. Accordingly, the user can set the amount of medication that they wish to administer to themselves. The dose adjustor 21 is mechanically connected and controlled by the dose adjustment dial 32. Thus, the dose adjustor 21 physically alters the amount of medication being administered.

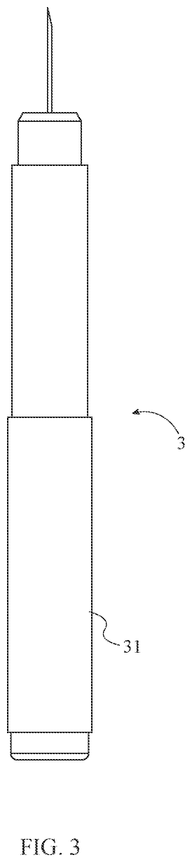

In reference to FIG. 3, the outer casing 31 is tubular in shape. The outer casing 31 further comprises a dosage window 311 and an LED light 312. So, the dosage window 311 and LED light 312 are integrated within the outer casing 31 surface. The dose adjustment dial 32 is tubular in shape. As a result, the dose adjustment dial 32 fits within the outer casing 31. The dose adjustment dial 32 further comprises a plurality of numbers 321. The injector button 33 is positioned on the top surface of the dose adjustment dial 32. Consequently, the injector button 33 is easily accessible to the user. The dosage window 311 creates a cutout on the outer surface at the proximal end of the outer casing 31. Accordingly, the dosage window 311 allows the user to see into the outer casing 31. The dosage window 311 reveals one of the plurality of numbers 321 of the dose adjustment dial 32. Thus, the user can observe which number the dose adjustment dial 32 is set to, which corresponds with a dose of the medicine within the medication cartridge 2. The LED light 312 is secured along the proximal end of the dosage window 311. So, the LED light 312 does not interfere with viewing the plurality of numbers 321. The LED light 312 illuminates one of the plurality of numbers 321 of the dose adjustment dial 32. As a result, the LED light 312 highlights the plurality of numbers 321 indicating which number is selected to the user.

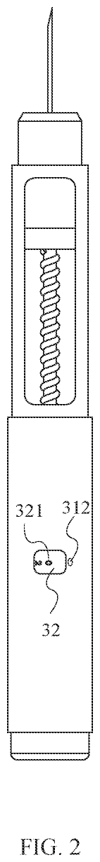

As shown in FIG. 2, the dose adjustment dial 32 rotates about the central axis of the dose adjustment dial 32. Consequently, the dose adjustment dial 32 rotates within the outer casing 31. The dose adjustment dial 32 rotates to mechanically move the dose adjustor 21. Accordingly, the dose adjustor 21 rotates in sync with the dose adjustment dial 32. The plurality of numbers 321 is scribed along the outer surface of the dose adjustment dial 32 that traverses longitudinally. Thus, the plurality of numbers 321 increases numerically and are evenly spaced out along the outer surface of the dose adjustment dial 32. The dose adjustment dial 32 slidably engages with the inner surface of the outer casing 31. So, the dose adjustment dial 32 rotates in place within the outer casing 31. The plurality of numbers 321 contains a phosphor substance. The plurality of numbers 321 glows in low light conditions. As a result, the plurality of numbers 321 can easily be read by the user within low light conditions to ensure that they are administering the proper amount of medicine.

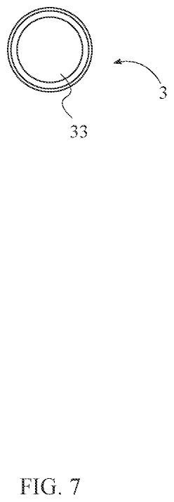

In reference to FIG. 7, the injector button 33 is mechanically connected to the pen body 3. Consequently, the injector button 33 slides in and out of the pen body 3. The injector button 33 compresses when the injector button 33 receives an external surface force. Accordingly, the injector button 33 returns to its original decompressed position when no external force is acting upon it. The injector button 33 releases the liquid medication from the medication cartridge 2 when the injector button 33 is compressed. Thus, the injector button 33 controls the release of the liquid medication.

Although the invention has been explained in relation to its preferred embodiment, it is to be understood that many other possible modifications and variations can be made without departing from the spirit and scope of the invention as hereinafter claimed.

What is claimed is:

1. An insulin self-injection pen comprising:
a needle;
a medication cartridge;
a pen body;
the medication cartridge comprising a dose adjustor;
the pen body comprising an outer casing, a dose adjustment dial and an injector button;
the needle securing to the distal end of the medication cartridge;
the medication cartridge being positioned within the pen body;
the pen body encompassing around the medication cartridge and needle; and
the needle extending outwards from the pen body.

2. The insulin self-injection pen as claimed in claim 1 comprising:
the needle being a long slender cylindrical shape; and
the needle transferring a liquid medication into the body of a user.

3. The insulin self-injection pen as claimed in claim 1 comprising:
the proximal end of the medication cartridge being attached to the pen body;
the medication cartridge being interchangeable within the pen body; and
the medication cartridge being secured within the pen body.

4. The insulin self-injection pen as claimed in claim 2 comprising:
the dose adjustor mechanically adjusting the quantity of medication being injected into the body of the user; and
the dose adjustor being mechanically connected and controlled by the dose adjustment dial.

5. The insulin self-injection pen as claimed in claim 1 comprising:
the outer casing being tubular in shape;
the outer casing further comprising a dosage window and an LED light;
the dose adjustment dial being tubular in shape;
the dose adjustment dial further comprising a plurality of numbers; and
the injector button being positioned on the top surface of the dose adjustment dial.

6. The insulin self-injection pen as claimed in claim 5 comprising:
the dosage window creating a cutout on the outer surface at the proximal end of the outer casing;
the dosage window revealing one of the plurality of numbers of the dose adjustment dial;
the LED light being secured along the proximal end of the dosage window; and
the LED light illuminating one of the plurality of numbers of the dose adjustment dial.

7. The insulin self-injection pen as claimed in claim 5 comprising:
the dose adjustment dial rotating about the central axis of the dose adjustment dial;
the dose adjustment dial rotating to mechanically move the dose adjustor;
the plurality of numbers being scribed along the outer surface of the dose adjustment dial that traverse longitudinally; and
the dose adjustment dial slidably engaging with the inner surface of the outer casing.

8. The insulin self-injection pen as claimed in claim 6 comprising:
the plurality of numbers containing a phosphor substance; and
the plurality of numbers glowing in low light conditions.

9. The insulin self-injection pen as claimed in claim 5 comprising:
the injector button being mechanically connected to the pen body;
the injector button compressing when receiving an external surface force; and
the injector button releasing the liquid medication from the medication cartridge when the injector button is compressed.

10. An insulin self-injection pen comprising:
a needle;
a medication cartridge;
a pen body;
the medication cartridge comprising a dose adjustor;
the pen body comprising an outer casing, a dose adjustment dial and an injector button;
the needle securing to the distal end of the medication cartridge;
the medication cartridge being positioned within the pen body;
the pen body encompassing around the medication cartridge and needle;
the needle extending outwards from the pen body;
the needle being a long slender cylindrical shape;
the needle transferring a liquid medication into the body of a user;
the proximal end of the medication cartridge being attached to the pen body;
the medication cartridge being interchangeable within the pen body; and
the medication cartridge being secured within the pen body.

11. The insulin self-injection pen as claimed in claim 10 comprising:
the dose adjustor mechanically adjusting the quantity of medication being injected into the body of the user; and
the dose adjustor being mechanically connected and controlled by the dose adjustment dial.

12. The insulin self-injection pen as claimed in claim 10 comprising:
the outer casing being tubular in shape;
the outer casing further comprising a dosage window and an LED light;
the dose adjustment dial being tubular in shape;
the dose adjustment dial further comprising a plurality of numbers; and
the injector button being positioned on the top surface of the dose adjustment dial.

13. The insulin self-injection pen as claimed in claim 12 comprising:
the dosage window creating a cutout on the outer surface at the proximal end of the outer casing;
the dosage window revealing one of the plurality of numbers of the dose adjustment dial;
the LED light being secured along the proximal end of the dosage window; and
the LED light illuminating one of the plurality of numbers of the dose adjustment dial.

14. The insulin self-injection pen as claimed in claim 12 comprising:
the dose adjustment dial rotating about the central axis of the dose adjustment dial;
the dose adjustment dial rotating to mechanically move the dose adjustor;
the plurality of numbers being scribed along the outer surface of the dose adjustment dial that traverse longitudinally; and
the dose adjustment dial slidably engaging with the inner surface of the outer casing.

15. The insulin self-injection pen as claimed in claim 14 comprising:
the plurality of numbers containing a phosphor substance; and
the plurality of numbers glowing in low light conditions.

16. The insulin self-injection pen as claimed in claim 12 comprising:
the injector button being mechanically connected to the pen body;
the injector button compressing when receiving an external surface force; and
the injector button releasing the liquid medication from the medication cartridge when the injector button is compressed.

17. An insulin self-injection pen comprising:
button;
a needle;
a medication cartridge;
a pen body;
the medication cartridge comprising a dose adjustor;
the pen body comprising an outer casing, a dose adjustment dial and an injector button;
the needle securing to the distal end of the medication cartridge;
the medication cartridge being positioned within the pen body;
the pen body encompassing around the medication cartridge and needle;
the needle extending outwards from the pen body;
the needle being a long slender cylindrical shape;
the needle transferring a liquid medication into the body of a user;
the proximal end of the medication cartridge being attached to the pen body;
the medication cartridge being interchangeable within the pen body;
the medication cartridge being secured within the pen body;
the dose adjustor mechanically adjusting the quantity of medication being injected into the body of the user;
the dose adjustor being mechanically connected and controlled by the dose adjustment dial;
the outer casing being tubular in shape;
the outer casing further comprising a dosage window and an LED light;
the dose adjustment dial being tubular in shape;
the dose adjustment dial further comprising a plurality of numbers; and
the injector button being positioned on the top surface of the dose adjustment dial.

18. The insulin self-injection pen as claimed in claim 17 comprising:
the dosage window creating a cutout on the outer surface at the proximal end of the outer casing;
the dosage window revealing one of the plurality of numbers of the dose adjustment dial;
the LED light being secured along the proximal end of the dosage window; and
the LED light illuminating one of the plurality of numbers of the dose adjustment dial.

19. The insulin self-injection pen as claimed in claim 17 comprising:
5 the dose adjustment dial rotating about the central axis of the dose adjustment dial;
the dose adjustment dial rotating to mechanically move the dose adjustor;
the plurality of numbers being scribed along the outer surface of the dose adjustment dial that traverse longitudinally;
the dose adjustment dial slidably engaging with the inner surface of the outer casing;
the plurality of numbers containing a phosphor substance; and
the plurality of numbers glowing in low light conditions.

20. The insulin self-injection pen as claimed in claim 17 comprising:
the injector button being mechanically connected to the pen body;
the injector button compressing when receiving an external surface force; and
the injector button releasing the liquid medication from the medication cartridge when the injector button is compressed.
